(19) United States
(12) Patent Application Publication
(10) Pub. No.: US 2024/0091137 A1
(43) Pub. Date: Mar. 21, 2024

(54) WellMed

(71) Applicant: Valerie Marie Gregory, St. Louis, MO (US)

(72) Inventor: Valerie Marie Gregory, St. Louis, MO (US)

(21) Appl. No.: 17/945,404

(22) Filed: Sep. 15, 2022

(51) Int. Cl. A61K 9/00; A61K 31/19; A61K 36/54; A61K 36/752; A61K 47/10

(52) U.S. Cl. CPC A61K 9/0014; A61K 31/19; A61K 36/54; A61K 36/752; A61K 47/10

(57) ABSTRACT

WellMed is a topical analgesic spray applied to an affected area of the body for pain relief, with no known side effects. It contains all natural ingredients and therefore is a green medication. Because the medication is not taken by mouth, it bypasses the body's metabolism for amore targeted form of pain relief.

### (1) BACKGROUND OF THE INVENTION

WellMed is an analgesic medication for use to alleviate pain topically. This description does not have any reference information regarding the invention.

### (2) BRIEF SUMMARY OF THE INVENTION

WellMed is a pain medication applied topically designed to eliminate pain. It is applied to the affected area with signs of relief in 15-20 minutes. Because it is not ingested, meaning taken by mouth, it bypasses the body's metabolism and is targeted to the affected area with direct application, resulting in pain relief more readily. All ingredients used have no known side effects.

### (3) BRIEF DESCRIPTION OF THE DRAWINGS

(Not Applicable)

### (4) DETAILED DESCRIPTION OF THE INVENTION

WellMed is a pain killer in the form of a spray that may be applied to an area of the body topically for pain relief. The ingredients to make the medication are naturally found in nature. WellMed is made using the following ingredients:
2 cups of water
1 tablespoon baking soda
½cup white vinegar
½ slice lemon (strained)
2 tablespoons 70% alcohol
1 shredded Persea americana seed

Mix and stir. Let solution sit for 24 hours in a clean spray bottle. Spray on the affected area or dip a cotton ball in the solution and apply to the affected area.

The claimed invention is:

1. WellMed is a topical medication spray designed to eliminate pain with relief noticed in 15-20 minutes of application, with no known side effects.
